(19) United States
(12) Patent Application Publication
(10) Pub. No.: US 2026/0234532 A1
(43) Pub. Date: Aug. 13, 2026

(54) 3D CELL CULTURE SYSTEM WITH PUMPLESS PERFUSION

(71) Applicants: University of Florida Research Foundation, Inc., Gainesville, FL (US); Aurita Corporation, Gainesville, FL (US)

(72) Inventors: Jack Famiglietti, Gainesville, FL (US); Wallace Gregory Sawyer, Gainesville, FL (US); Ryan Smolchek, Gainesville, FL (US)

(21) Appl. No.: 19/104,090

(22) PCT Filed: Aug. 14, 2023
(86) PCT No.: PCT/US2023/072143

§ 371 Date: Feb. 14, 2025

Related U.S. Application Data
(60) Provisional application No. 63/371,424, filed on Aug. 15, 2022.

(51) Int. Cl. C12M 1/00; C12M 1/12; C12M 1/32; C12M 1/34; C12M 1/42

(52) U.S. Cl. CPC C12M 29/10; C12M 23/12; C12M 23/22; C12M 25/16; C12M 29/00; C12M 29/04; C12M 35/04; C12M 41/44

(57) ABSTRACT

Disclosed herein is a three dimensional (3D) culture system for high-throughput screening with pumpless perfusion. It is a two well flow system where one well has a membrane at the bottom and the other is open. Both wells are connected so the flow is forced in one direction. A dialysis filter membrane can act as a resistor for a system that has a resistance to flow from the 3D culture medium (which is difficult to control) and a resistance from the filter membrane (which is easy to control). The liquid culture media is added to the reservoir well and the 3D culture medium to the sample well. Media will flow through the membrane and the 3D culture medium at a perfusion velocity that is governed in part by the number and size of holes in the membrane.

### CROSS-REFERENCE TO RELATED APPLICATIONS

This application claims benefit of U.S. Provisional Application No. 63/371,424, filed Aug. 15, 2022, which is hereby incorporated herein by reference in its entirety.

### BACKGROUND

The printing or placement of biological samples (e.g., cells, cell layers, tissues) into a 3D support medium more accurately and reproducibly models cellular morphology, heterogeneity, and genetic profiles seen in vivo compared to conventional 2D culture.

Some existing 3D cell culture techniques rely on polymer scaffolds in which cells are seeded and allowed to adhere. Once the cells are adhered to the scaffold, perfusion of growth media can begin. This method has several disadvantages: (1) cell migration is limited or precluded, (2) cell environments are defined by the structure of the polymer scaffold, (3) the experimental setup is not time-effective, and (4) does not include optical access for microscopy. In addition, cell viability for existing 3D culture methods is generally limited to several days; the passive 3D support medium cannot efficiently expel cellular waste, leading to localized cytotoxic environments and subsequent cell death. Improved culture systems are needed to increase cell viability.

### SUMMARY

Disclosed herein is a three dimensional (3D) culture system for high-throughput screening with pumpless perfusion. It is a two well flow system where one well has a membrane at the bottom and the other is open. Both wells are connected so the flow is forced in one direction. As disclosed herein a dialysis filter membrane can act as a resistor for a system that has a resistance to flow from the 3D culture medium (which is difficult to control) and a resistance from the filter membrane (which is easy to control). The liquid culture media is added to the reservoir well and the 3D culture medium to the sample well. Samples are suspended in the 3D culture medium prior to media perfusion. Media will flow through the membrane and the 3D culture medium at a perfusion velocity that is governed in part by the number and size of holes in the membrane. Liquid medium can be added to the reservoir well over time as well as liquid removed from the sample well so that perfusion can continue, i.e., since it will slow and eventually stop as the levels are equalized.

Therefore, disclosed herein is a multi-well plate system involving a sample well filled with a three-dimensional (3D) cell growth medium, and a liquid medium reservoir fluidly connected to the sample well by a filter membrane positioned at the bottom of the sample well, wherein filling the liquid medium reservoir to a first volume with liquid medium establishes a hydrostatic pressure that pushes the liquid medium from the liquid medium reservoir, vertically through the filter membrane, and into the sample well where it permeates the 3D cell growth medium. This vertical flow upwards through the filter membrane can provide a uniform perfusion velocity through the sample well since the hydrostatic pressure pushing the liquid medium is equilibrated by the resistance of the membrane filter. The hydrostatic pressure is set by the depth within the fluid, and the membrane reacts to this pressure with internal tension, while the fluid is allowed to pass through. Because the membrane is perpendicular with respect to gravity, all points on the membrane experiencing the same hydrostatic pressure, there is a uniform pressure gradient across the membrane at all points and this is what creates a uniform fluid velocity.

In some embodiments, the liquid medium reservoir is filled to the first volume, the filter membrane has a pore size configured to produce a perfusion velocity of the liquid medium through the 3D cell growth medium at least 100, 110, 120, 130, 140, 150, 160, 170, 180, 190, 200, 210, 220, 230, 240, or 250 nm/s. This tunable resistance allows the user to control the perfusion velocity. In some embodiments, the upper limit of the perfusion rate is determined by the 3D culture medium being used. For example, in some embodiments, the perfusion velocity of the liquid medium through the 3D cell growth medium is 100 nm/s to 5 pm/s, including 100, 110, 120, 130, 140, 150, 160, 170, 180, 190, 200, 210, 220, 230, 240, or 250 nm/s to 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, or 5.0 μm/s.

In some embodiments, the perfusion velocity can be tuned to oppose gravity in the 3D culture medium. The fluid velocity is calculated via an equation as shown in FIG. 8. In the way that formula was derived, if the velocity value is positive, the fluid moved upward, against gravity. If it is negative, it moves downward along the direction of gravity. It is based off a simple model using two fluidly connected chamber with equal cross sectional areas.

In some embodiments, the pore size is from 100 nm (0.1μm) to 10μm, including 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, or 10 μm.

In some embodiments, the system contains a means for transferring the liquid medium from the sample well to the liquid medium reservoir. There are a number of automated fluid handling systems available that could do this. Essentially robotic pipettors that precisely place a pipette tip into a well, collect a volume of media, and place it somewhere else. This could also be done manually.

In some embodiments, the system contains an overflow lip or drain in the sample well configured to empty the liquid medium into a collection container, thereby maintaining a constant sample well volume. In some embodiments, the system contains a means for transferring the liquid medium from the collection container to the liquid medium reservoir. For example, in some embodiments, the means for transferring the liquid medium from the collection container to the liquid medium reservoir is a peristaltic pump. This could be done by connecting the peristaltic pump between the two chambers, or by use of any other very slow flow pump, or as mentioned above, by use of liquid handling devices.

In some embodiments, the system contains a means for monitoring the volume of liquid medium in the liquid medium reservoir configured to trigger the means for transferring the liquid medium from the collection container to the liquid medium reservoir when the volume of liquid medium in the liquid medium reservoir drops below the first volume. For example, the system can contain an optical sensor or float switch. This could also be done by eye as well. It could further be monitored with a pressure sensor within one of the liquid chambers. Another method would be to have a sensor monitoring fluid velocity, as fluid velocity and fluid height difference are directly linked.

In some embodiments, the system contains an array of isolated sample wells that are all fluidly connected to the same liquid medium reservoir or to a separate liquid medium reservoir.

In some embodiments, the bottom of the sample well is optically transparent.

In some embodiments, the 3D cell culture medium is a hydrogel.

In some embodiments, the 3D cell culture medium comprises a plurality of hydrogel particles and a liquid cell culture medium, wherein the hydrogel particles are swelled with the liquid cell culture medium to form a granular gel. For example, the 3D cell culture medium can have a yield stress such that the cell growth medium undergoes a phase change from a first solid phase to a second liquid phase upon application of a shear stress greater than the yield stress. In some embodiments, the yield stress is on the order of 10 Pa. In some embodiments, the concentration of hydrogel particles is between 0.05% to about 1.0% by weight. In some embodiments, the hydrogel particles have a size between about 0.1 μm to about 100 μm when swollen with the liquid cell culture medium.

In some embodiments, the 3D cell culture medium is a liquid medium.

In some embodiments, the system contains a plurality of cells are disposed in the 3D cell culture medium.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 is a perspective view of an embodiment multi-well plate with gravity driven perfusion.

FIG. 2 is an exploded view of an embodiment multi-well plate with gravity driven perfusion.

FIG. 3 is a top down view of an embodiment multi-well plate with gravity driven perfusion.

FIG. 4 is a cross section view of wells in an embodiment multi-well plate with gravity driven perfusion.

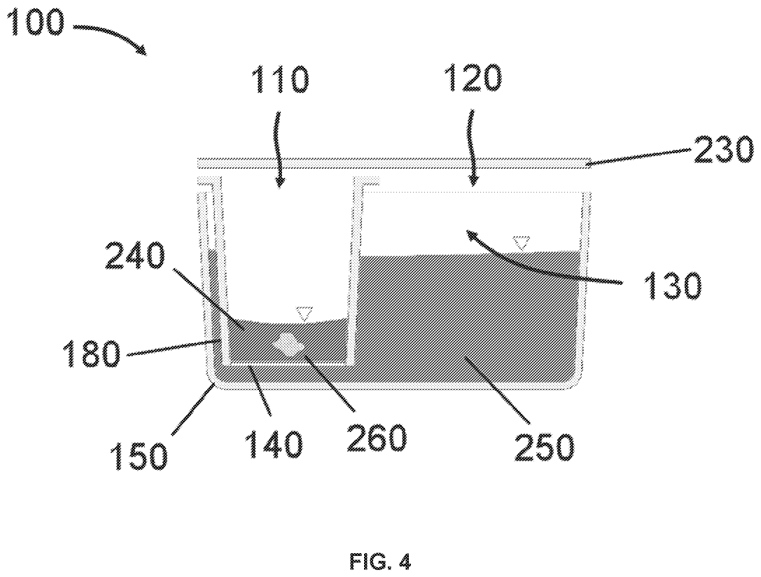

FIG. 5 illustrates a multi-well perfusion setup protocol embodiment for experiment setup and maintenance with gravity fed perfusion.

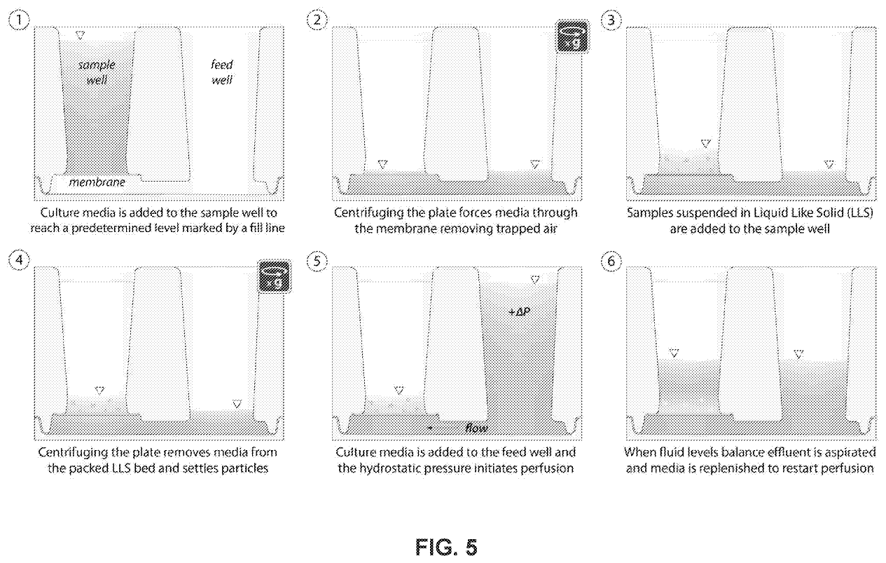

FIG. 6 shows passive perfusion with a 0.2 μ pore perfusion column.

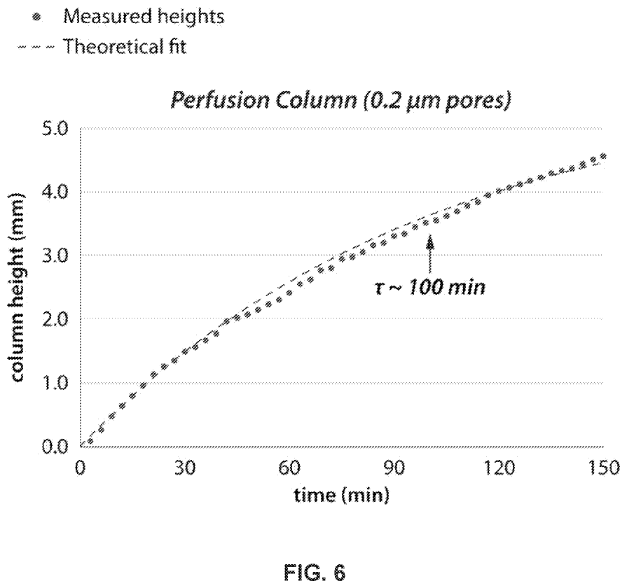

FIG. 7 shows passive perfusion with a 0.2 μ pore perfusion column with a liquid like solid (LLS) support medium.

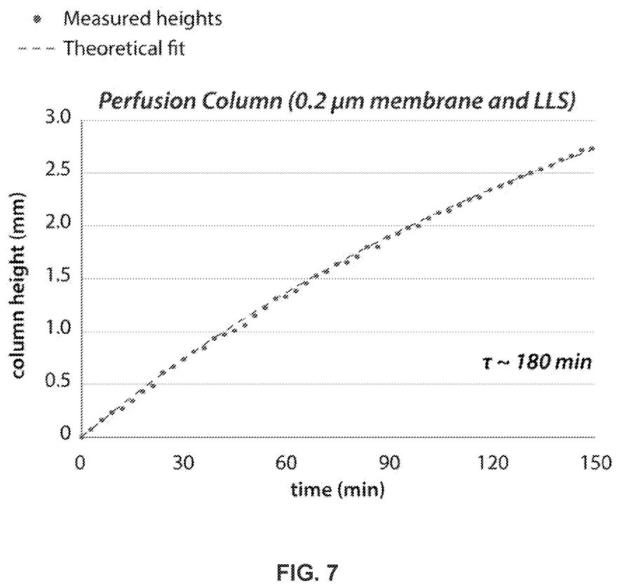

FIG. 8 shows perfusion interstitial velocity with a 0.2 μ pore perfusion column with LLS support medium.

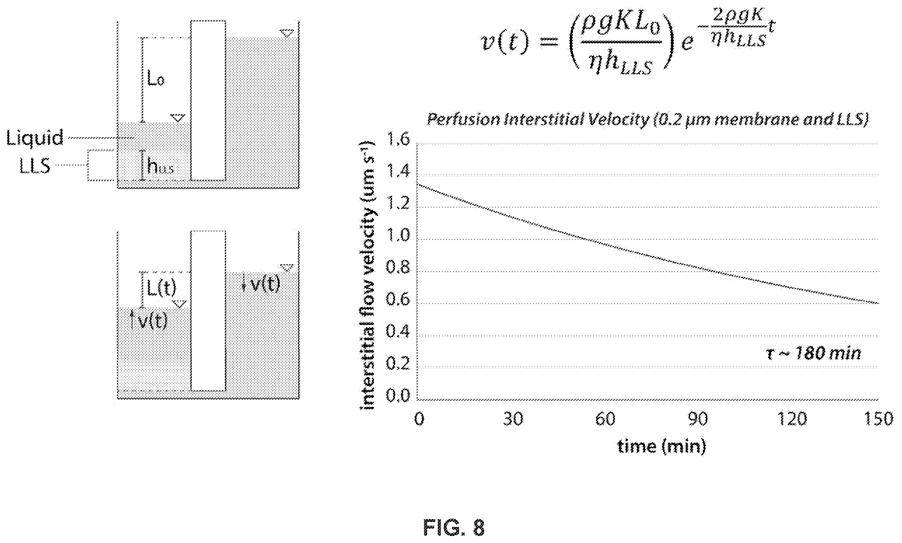

FIG. 9 is a cross section view of wells in an embodiment multi-well plate with gravity driven perfusion.

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of chemistry, biology, and the like, which are within the skill of the art.

The following examples are put forth so as to provide those of ordinary skill in the art with a complete disclosure and description of how to perform the methods and use the probes disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

Before the embodiments of the present disclosure are described in detail, it is to be understood that, unless otherwise indicated, the present disclosure is not limited to particular materials, reagents, reaction materials, manufacturing processes, or the like, as such can vary. It is also to be understood that the terminology used herein is for purposes of describing particular embodiments only, and is not intended to be limiting. It is also possible in the present disclosure that steps can be executed in different sequence where this is logically possible.

It must be noted that, as used in the specification and the appended claims, the singular forms “a,” “an,” and “the” include plural referents unless the context clearly dictates otherwise.

### 3D Culture System

Turning now to the figures, specific non-limiting embodiments of bioreactor system are described in more detail.

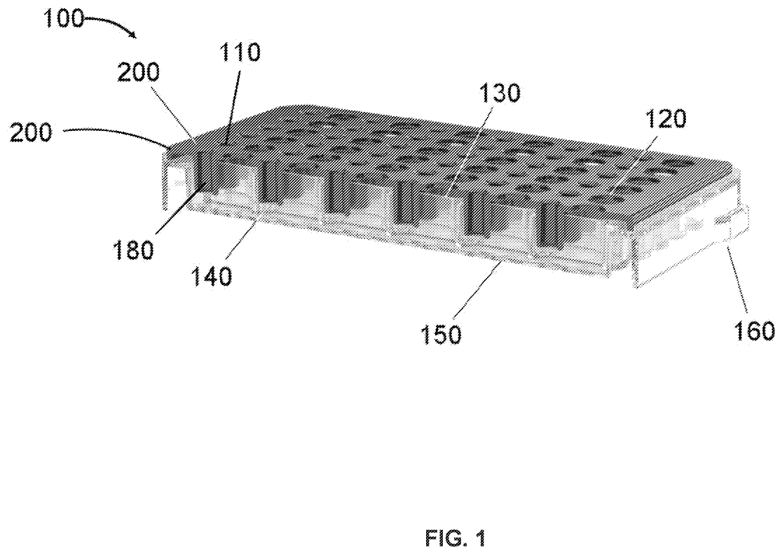

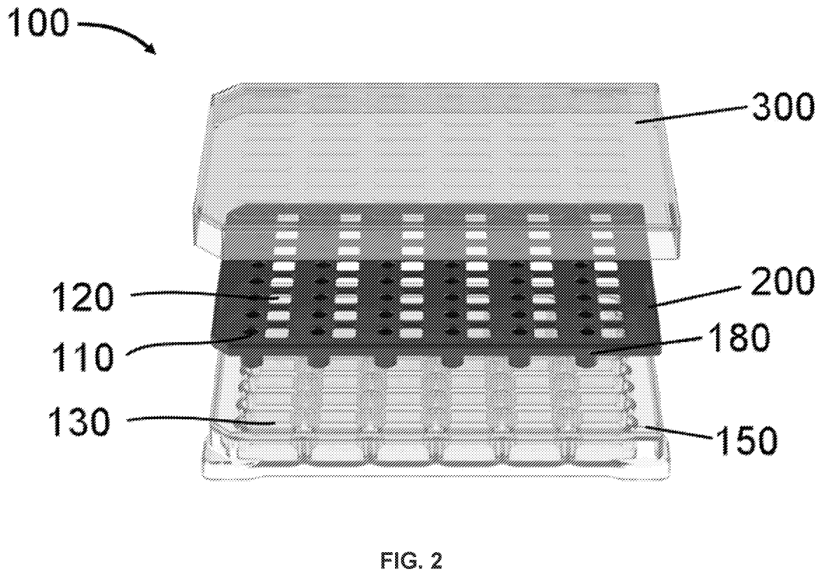

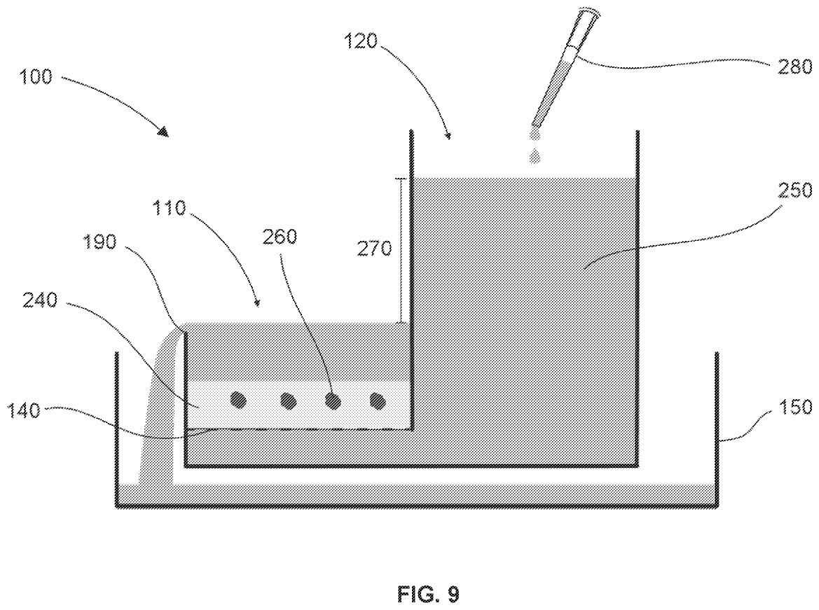

As shown in FIGS. 1 to 3 and 9, a multi-well plate system with gravity driven perfusion 100 is disclosed that includes a reservoir plate 150 and a sample well plate 200 wherein the sample well plate 200 can be positioned on top of the reservoir plate 150. The reservoir plate 150 can have a liquid media reservoir/wells 130, i.e., configured as a single reservoir or a plurality of feed wells. The sample well plate 200 can have one or more sample wells 180 such that when the sample well plate 200 is positioned on top of the reservoir plate 150, the sample well becomes fluidly connected to the liquid media reservoir/wells 130, separated by a filter membrane 140. In some embodiments, the filter membrane 140 is positioned at the bottom of the sample wells 180.

The reservoir plate 150 can have one or more media ports 120 for filling the liquid media reservoir/wells 130. The sample well plate 200 can have one or more sample ports 110 for adding a sample to the sample wells 180.

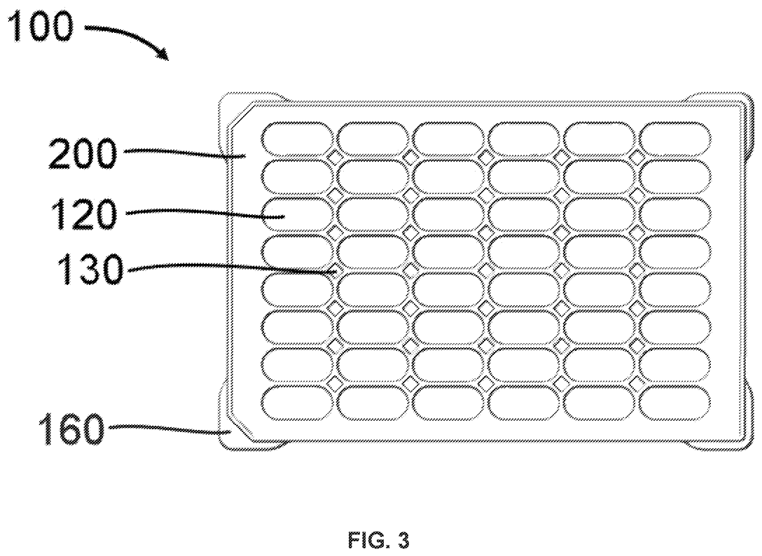

As shown in FIG. 3, the sample wells 180 can be filled with a 3D support medium 240 disclosed herein, and the liquid media reservoir/wells 130 can be filed with a liquid culture media 250. The liquid media reservoir/wells 130 are in some embodiments filled to a reservoir height that is greater than the height of the 3D support medium 240 in the sample wells 180. This fluid height difference 270 generates a hydrostatic pressure. This fluid height difference 270 can be maintained by a steady supplier 280 of liquid culture media 250. Examples for a media supplier 280 include a pipette, pump, or the equivalent.

In some embodiments, the filter membrane 140 sustains the hydrostatic pressure generated from the height difference of the fluid levels in the liquid media reservoir/wells 130 and sample wells 180. The filter membrane can hold the hydrostatic pressure in tension in the membrane itself while allowing liquid culture media 250 to flow through it and thus through the 3D support medium 240. In some embodiments, the 3D support medium 240 has essentially zero friction with the walls of sample wells 180, so if the membrane were not present the hydrostatic pressure would push the 3D support medium 240 up and away and disrupt the culture. Therefore, the filter membrane 140 can in some embodiments be selected to provide a force balance and not solely as a flow controller.

In some embodiments, the sample ports 110 have a lip 190 that allows for overflow of the liquid culture media 250 into the reservoir plate 150.

In some embodiments, a biological sample 260 is positioned within the 3D support medium 240. The perfusion of liquid culture media 250 from the hydrostatic pressure can support cell culture without the need for a pump. In some embodiments, liquid culture media 250 can be removed (e.g. aspirated) from the sample wells 180 and added to the liquid media reservoir/wells 130 to maintain the hydrostatic pressure until the culture is complete.

In some embodiments, the disclosed sample wells can be used for culturing cells (such as tumor samples) in a 3D cell growth medium, which allows the nutrient media to perfuse through the system without disturbing the cellular environment. The wells of the bioreactor system are preferably arrayed in columns and rows.

As described below, the 3D cell growth medium may include a thixotropic or yield stress material, or any material suitable for temporary phase changing. In some examples, the thixotropic or yield stress material may include a soft granular gel. The soft granular gel may be made from polymeric hydrogel particles swelled with a liquid cell culture medium. Depending on the particular embodiment, the hydrogel particles may be between 0.5 μm and 50 μm in diameter, between about 1 μm and 10 μm in diameter, or about 5 μm in diameter when swelled.

### Filter material

The disclosed system contains one or more filter membranes (e.g. dialysis membranes) that allow the liquid media to perfuse through the system by hydrostatic pressure. In some embodiments, the filter membrane is a 0.2 μm polycarbonate track-etched (PCTE) membrane.

The membrane material could be made of essentially any material that is not soluble in water. Some other examples include PEEK, polyethylene, polystyrene, nylon, polyethersulfone, polyester, polypropylene, polytetrafluoride (PTFE), silver, aluminum oxide, cellulose.

The pore size will vary with the required application, which will require various flow velocities. Another important parameter to consider here is the pore density on a given membrane. The number of pores in a given region will linearly affect the resistance of the membrane. This parameter may be more easily characterized as open area percentage.

In some embodiments, the pore size is from 0.1 μm to 10 μm, including 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, or 10 μm.

In some embodiments, the filter membrane may be composed of a nanoporous sheet membranes of material polycarbonate, nylon, or various other materials which produce the same effect while remaining biocompatible or inert.

### 3D Growth Medium

There are several biomaterials that can be used in tissue engineering and cell delivery, with most of these materials being considered “hydrogels”, or water-containing gels. The requirements for these materials are 1) biocompatibility: cells must be able to be combined with the materials, often throughout the material, and remain viable and functional, and 2) in most cased the hydrogels must also facilitate migration, proliferation and differentiation of the embedded and endogenous, cells. There are additional constraints for maintaining “printability” of hydrogels, while still keeping initial requirements of biocompatibility and migration/function. These are outlined in the review by Malda, J, et al, Adv. Mater. 2013, 25, 5011-5028. In addition, many commercial sources of hydrogels that can be used for bio-printing are described by Murphy S V, Skardal A, Atala A. 2013. Evaluation of hydrogels for bio-printing applications. J Biomed Mater Res Part A 2013:101A:272-284, including Collagen Type I, Collagen/Fibrin, Fibrin, Extracel™ hydrogel, Extracel™ UV, Tyramine substituted hyaluronic acid (TS-NaHy), Corgel™, Methylcellulose-Hyaluronan (MC-HA), Chitosan, Chitosan/Collagen, Alginate, Alginate/Gelatin, and Polyethylene Glycol Diacrylate (PEGDA).

Common materials used in tissue engineering and bioprinting are alginates, collagens, fibrins, fibrinogens, polyethylene glycols (PEGs), agar, agarose, chitosan, hyaluronan, methacrylamide, gelatins, pluronics, matrigel, methylcellulose, and PEG-DA (diacrylate). These materials are often not used simply alone, but are often mixed together to combine properties (e.g. gelling properties of alginates or PEG-DA with cell adhesion abilities of collagens/fibrins) to make new compositions. Furthermore, new hydrogels can be designed from the ground up to account for these properties (Guvendiren and Burdick, Current Opinion in Biotechnology 2013, 24:841-846).

In some embodiments, the 3D support medium is a liquid medium. In some embodiments, cells are cultured directly on the membrane and the fluid flow is used as a way to continuously flush waste away or to dose them with something or otherwise. This would be a way to use the system without any 3D support medium other than liquid.

In some embodiments, the 3D support medium is a liquid-like solid (LLS) three-dimensional (3D) medium. For example, in some embodiments, the 3D support medium is the LLS medium disclosed in WO2016182969A1, which is incorporated by reference in its entirety for the description of how to make and uses this LLS medium.

Briefly, the 3D cell growth medium may comprise hydrogel particles dispersed in a liquid cell growth medium. Any suitable liquid cell growth medium may be used; a particular liquid cell growth medium may be chosen depending on the types of cells which are to be placed within the 3D cell growth medium. For example, suitable cell growth medium may be human cell growth medium, murine cell growth medium, bovine cell growth medium or any other suitable cell growth medium. Depending on the particular embodiment, hydrogel particles and liquid cell growth medium may be combined in any suitable combination. For example, in some embodiments, a 3D cell growth medium comprises approximately 0.5% to 1% hydrogel particles by weight.

In accordance with some embodiments, the hydrogel particles may be made from a bio-compatible polymer.

The hydrogel particles may swell with the liquid growth medium to form a granular gel material. Depending on the particular embodiment, the swollen hydrogel particles may have a characteristic size at the micron or submicron scales. For example, in some embodiments, the swollen hydrogel particles may have a size between about 0.1 μm and 100 μm. Furthermore, a 3D cell growth medium may have any suitable combination of mechanical properties, and in some embodiments, the mechanical properties may be tuned via the relative concentration of hydrogel particles and liquid cell growth medium. For example, a higher concentration of hydrogel particles may result in a 3D growth medium having a higher elastic modulus and/or a higher yield stress.

According to some embodiments, the 3D cell growth medium may be made from materials such that the granular gel material undergoes a temporary phase change due to an applied stress (e.g. a thixotropic or “yield stress” material). Such materials may be solids or in some other phase in which they retain their shape under applied stresses at levels below their yield stress. At applied stresses exceeding the yield stress, these materials may become fluids or in some other more malleable phase in which they may alter their shape. When the applied stress is removed, yield stress materials may become solid again. Stress may be applied to such materials in any suitable way. For example, energy may be added to such materials to create a phase change. The energy may be in any suitable form, including mechanical, electrical, radiant, or photonic, etc.

Regardless of how cells are placed in the medium, the yield stress of the yield stress material may be large enough to prevent yielding due to gravitational and/or diffusional forces exerted by the cells such that the position of the cells within the 3D growth medium may remain substantially constant over time. As described in more detail below, placement and/or retrieval of groups of cells may be done manually or automatically.

A yield stress material as described herein may have any suitable mechanical properties. For example, in some embodiments, a yield stress material may have an elastic modulus between approximately 1 Pa and 1000 Pa when in a solid phase or other phase in which the material retains its shape under applied stresses at levels below the yield stress. In some embodiments, the yield stress required to transform a yield stress material to a fluid-like phase may be between approximately 1 Pa and 1000 Pa. In some embodiments, the yield stress may be on the order of 10 Pa, such as 10 Pa +/−25%. When transformed to a fluid-like phase, a yield stress material may have a viscosity between approximately 1 Pa s and 10,000 Pa s. However, it should be understood that other values for the elastic modulus, yield stress, and/or viscosity of a yield stress material are also possible, as the present disclosure is not so limited.

A group of cells may be placed in a 3D growth medium made from a yield stress material via any suitable method. For example, in some embodiments, cells may be injected or otherwise placed at a particular location within the 3D growth medium with a syringe, pipette, or other suitable placement or injection device. In some embodiments an array of automated cell dispensers may be used to inject multiple cell samples into a container of 3-D growth medium. Movement of the tip of a placement device through the 3D growth medium may impart a sufficient amount of energy into a region around the tip to cause yielding such that the placement tool may be easily moved to any location within the 3D growth medium. In some instances, a pressure applied by a placement tool to deposit a group of cells within the 3D growth medium may also be sufficient to cause yielding such that the 3D growth medium flows to accommodate the group of cells. Movement of a placement tool may be performed manually (e.g. “by hand”), or may performed by a machine or any other suitable mechanism.

In some embodiments, multiple independent groups of cells may be placed within a single volume of a 3D cell growth medium. For example, a volume of 3D cell growth medium may be large enough to accommodate at least 2, at least 5, at least 10, at least 20, at least 50, at least 100, at least 1000, or any other suitable number of independent groups of cells. Alternatively, a volume of 3D cell growth medium may only have one group of cells. Furthermore, it should be understood that a group of cells may comprise any suitable number of cells, and that the cells may of one or more different types.

Depending on the particular embodiment, groups of cells may be placed within a 3D cell growth medium according to any suitable shape, geometry, and/or pattern. For example, independent groups of cells may be deposited as spheroids, and the spheroids may be arranged on a 3D grid, or any other suitable 3D pattern. The independent spheroids may all comprise approximately the same number of cells and be approximately the same size, or alternatively different spheroids may have different numbers of cells and different sizes. In some embodiments, cells may be arranged in shapes such as embryoid or organoid bodies, tubes, cylinders, toroids, hierarchically branched vessel networks, high aspect ratio objects, thin closed shells, or other complex shapes which may correspond to geometries of tissues, vessels or other biological structures.

According to some embodiments, a 3D cell growth medium made from a yield stress material may enable 3D printing of cells to form a desired pattern in three dimensions. For example, a computer-controlled injector tip may trace out a spatial path within a 3D cell growth medium and inject cells at locations along the path to form a desired 3D pattern or shape. Movement of the injector tip through the 3D cell growth medium may impart sufficient mechanical energy to cause yielding in a region around the injector tip to allow the injector tip to easily move through the 3D cell growth medium, and also to accommodate injection of cells. After injection, the 3D cell growth medium may transform back into a solid-like phase to support the printed cells and maintain the printed geometry. However, it should be understood that 3D printing techniques are not required to use a 3D growth medium as described herein.

According to some embodiments, a 3D cell growth medium may be prepared by dispersing hydrogel particles in a liquid cell growth medium. The hydrogel particles may be mixed with the liquid cell growth medium using a centrifugal mixer, a shaker, or any other suitable mixing device. During mixing, the hydrogel particles may swell with the liquid cell growth medium to form a material which is substantially solid when an applied shear stress is below a yield stress, as discussed above. After mixing, entrained air or gas bubbles introduced during the mixing process may be removed via centrifugation, agitation, or any other suitable method to remove bubbles from 3D cell growth medium.

In some embodiments, preparation of a 3D cell growth medium may also involve buffering to adjust the pH of a hydrogel particle and liquid cell growth medium mixture to a desired value. For example, some hydrogel particles may be made from polymers having a predominantly negative charge which may cause a cell growth medium to be overly acidic (have a pH which is below a desired value). The pH of the cell growth medium may be adjusted by adding a strong base to neutralize the acid and raise the pH to reach the desired value. Alternatively, a mixture may have a pH that is higher than a desired value; the pH of such a mixture may be lowered by adding a strong acid. According to some embodiments, the desired pH value may be in the range of about 7.0 to 7.4, or, in some embodiments 7.2 to 7.6, or any other suitable pH value which may, or may not, correspond to in vivo conditions. The pH value, for example may be approximately 7.4. In some embodiments, the pH may be adjusted once the dissolved CO2 levels are adjusted to a desired value, such as approximately 5%.

Yield stress can be measured by performing a strain rate sweep in which the stress is measured at many constant strain rates. Yield stress can be determined by fitting these data to a classic Herschel-Bulkley model (σ=σ_{y}+k\dot{γ}ⁿ). (b) To determine the elastic and viscous moduli of non-yielded LLS media, frequency sweeps at 1% strain can be performed. The elastic and viscous moduli remain flat and separated over a wide range of frequency, behaving like a Kelvin-Voigt linear solid with damping. Together, these rheological properties demonstrate that a smooth transition between solid and liquid phases occurs with granular microgels, facilitating their use as a 3D support matrix for cell printing, culturing, and assaying.

An example of a hydrogel with which some embodiments may operate is a carbomer polymer, such as Carbopol®. Carbomer polymers may be polyelectrolytic, and may comprise deformable microgel particles. Carbomer polymers are particulate, high-molecular-weight crosslinked polymers of acrylic acid with molecular weights of up to 3-4 billion Daltons. Carbomer polymers may also comprise co-polymers of acrylic acid and other aqueous monomers and polymers such as poly-ethylene-glycol.

While acrylic acid is a common primary monomer used to form polyacrylic acid the term is not limited thereto but includes generally all α-β unsaturated monomers with carboxylic pendant groups or anhydrides of dicarboxylic acids and processing aids as described in U.S. Pat. No. 5,349,030. Other useful carboxyl containing polymers are described in U.S. Pat. No. 3,940,351, directed to polymers of unsaturated carboxylic acid and at least one alkyl acrylic or methacrylic ester where the alkyl group contains 10 to 30 carbon atoms, and U.S. Pat. Nos. 5,034,486; 5,034,487; and 5,034,488; which are directed to maleic anhydride copolymers with vinyl ethers. Other types of such copolymers are described in U.S. Pat. No. 4,062,817 wherein the polymers described in U.S. Pat. No. 3,940,351 contain additionally another alkyl acrylic or methacrylic ester and the alkyl groups contain 1 to 8 carbon atoms. Carboxylic polymers and copolymers such as those of acrylic acid and methacrylic acid also may be cross-linked with polyfunctional materials as divinyl benzene, unsaturated diesters and the like, as is disclosed in U.S. Pat. Nos. 2,340,110; 2,340,111; and 2,533,635. The disclosures of all of these U.S. Patents are hereby incorporated herein by reference for their discussion of carboxylic polymers and copolymers that, when used in polyacrylic acids, form yield stress materials as otherwise disclosed herein. Specific types of cross-linked polyacrylic acids include carbomer homopolymer, carbomer copolymer and carbomer interpolymer monographs in the U.S. Pharmocopia 23 NR 18, and Carbomer and C10-30alkylacrylate crosspolymer, acrylates crosspolymers as described in PCPC International Cosmetic Ingredient Dictionary & Handbook, 12th Edition (2008).

Carbomer polymer dispersions are acidic with a pH of approximately 3. When neutralized to a pH of 6-10, the particles swell dramatically. The addition of salts to swelled Carbomer can reduce the particle size and strongly influence their rheological properties. Swelled Carbomers are nearly refractive index matched to solvents like water and ethanol, making them optically clear. The original synthetic powdered Carbomer was trademarked as Carbopol® and commercialized in 1958 by BF Goodrich (now known as Lubrizol), though Carbomers are commercially available in a multitude of different formulations.

Hydrogels may include packed microgels—microscopic gel particles, ~5μm in diameter, made from crosslinked polymer. The yield stress of Carbopol® is controlled by water content. Carbopol® yield stress can be varied between roughly 1-1000 Pa. Thus, both materials can be tuned to span the stress levels that cells typically generate. As discussed above, while materials may have yield stresses in a range of 1-1000 Pa, in some embodiments it may be advantageous to use yield stress materials having yield stresses in a range of 1-100 Pa or 10-100 Pa. In addition, some such materials may have thixotropic times less than 2.5, less than 1.5 seconds, less than 1 second, or less than 0.5 seconds, and greater than 0.25 seconds or greater than 0.1 seconds, and/or thixotropic indexes less than 7, less than 6.5, or less than 5, and greater than 4, or greater than 2, or greater than 1.

### Liquid Medium

Liquid medium composition must be considered from two perspectives: basic nutrients (sugars, amino acids) and growth factors/cytokines. Co-culture of cells often allows reduction or elimination of serum from the medium due to production of regulatory macromolecules by the cells themselves. The ability to supply such macromolecular regulatory factors in a physiological way is a primary reason 3D perfused co-cultures are used. A serum-free medium supplemented with several growth factors suitable for long-term culture of primary differentiated hepatocytes has been tested and found to support co-culture of hepatocytes with endothelial cells. ES cells are routinely maintained in a totipotent state in the presence of leukemia inhibitory factor (LIF), which activates gp130 signaling pathways. Several medium formulations can support differentiation of ES cells, with different cytokine mixes producing distinct patterns of differentiation. Medium replacement rates can be determined by measuring rates of depletion of key sugars and amino acids as well as key growth factors/cytokines. If cell culture medium with sodium bicarbonate is used, the environmental control can be provided by e.g. placing the module with bioreactor/reservoir pairs into a CO₂ incubator.

### Cells

A variety of different cells can be applied to the 3D growth medium of the disclosed systems. In some embodiments, these are normal human cells or human tumor cells. The cells may be a homogeneous suspension or a mixture of cell types. The different cell types may be seeded onto and/or into the medium sequentially, together, or after an initial suspension is allowed to attach and proliferate (for example, endothelial cells, followed by liver cells). Cells can be obtained from cell culture or biopsy. Cells can be of one or more types, either differentiated cells, such as endothelial cells or parenchymal cells, including nerve cells, or undifferentiated cells, such as stem cells or embryonic cells. In one embodiment, the medium is seeded with a mixture of cells including endothelial cells, or with totipotent/pluripotent stem cells which can differentiate into cells including endothelial cells, which will form “blood vessels”, and at least one type of parenchymal cells, such as hepatocytes, pancreatic cells, or other organ cells.

Cells can be cultured initially and then used for screening of compounds for toxicity. Cells can also be used for screening of compounds having a desired effect. For example endothelial cells can be used to screen compounds which inhibit angiogenesis. Tumor cells can be used to screen compounds for anti-tumor activity. Cells expressing certain ligands or receptors can be used to screen for compounds binding to the ligands or activating the receptors. Stem cells can be seeded, alone or with other types of cells. Cells can be seeded initially, then a second set of cells introduced after the initial bioreactor tissue is established, for example, tumor cells that grow in the environment of liver tissue. The tumor cells can be studied for tumor cell behaviors or molecular events can be visualized during tumor cell growth. Cells can be modified prior to or subsequent to introduction into the apparatus. Cells can be primary tumor cells from patients for diagnostic and prognostic testing. The tumor cells can be assessed for sensitivity to an agent or gene therapy. Tumor cell sensitivity to an agent or gene therapy can be linked to liver metabolism of set agent or gene therapy. Cells can be stem or progenitor cells and the stem or progenitor cells be induced to differentiate by the mature tissue. Mature cells can be induced to replicate by manipulation of the flow rates or medium components in the system.

### Applications

The disclosed system has many different applications, such as identification of markers of disease; assessing efficacy of anti-cancer therapeutics; testing gene therapy vectors; drug development; screening; studies of cells, especially stem cells; studies on biotransformation, clearance, metabolism, and activation of xenobiotics; studies on bioavailability and transport of chemical agents across epithelial layers; studies on bioavailability and transport of biological agents across epithelial layers; studies on transport of biological or chemical agents across the blood-brain barrier; studies on acute basal toxicity of chemical agents; studies on acute local or acute organ-specific toxicity of chemical agents; studies on chronic basal toxicity of chemical agents; studies on chronic local or chronic organ-specific toxicity of chemical agents; studies on teratinogenicity of chemical agents; studies on genotoxicity, carcinogenicity, and mutagenicity of chemical agents; detection of infectious biological agents and biological weapons; detection of harmful chemical agents and chemical weapons; studies on infectious diseases; studies on the efficacy of chemical agents to treat disease; studies on the efficacy of biological agents to treat disease; studies on the optimal dose range of agents to treat disease; prediction of the response of organs in vivo to biological agents; prediction of the pharmacokinetics of chemical or biological agents; prediction of the pharmacodynamics of chemical or biological agents; studies concerning the impact of genetic content on response to agents; filter or porous material below microscale tissue may be chosen or constructed so as bind denatured, single-stranded DNA; studies on gene transcription in response to chemical or biological agents; studies on protein expression in response to chemical or biological agents; studies on changes in metabolism in response to chemical or biological agents; prediction of agent impact through database systems and associated models; prediction of agent impact through expert systems; and prediction of agent impact through structure-based models.

A number of embodiments of the invention have been described. Nevertheless, it will be understood that various modifications may be made without departing from the spirit and scope of the invention. Accordingly, other embodiments are within the scope of the following claims.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

1. A multi-well plate system, comprising
a) a sample well filled with a three-dimensional (3D) cell growth medium, and
b) a liquid medium reservoir fluidly connected to the sample well by a filter membrane positioned at the bottom of the sample well,
wherein filling the liquid medium reservoir to a first volume with liquid medium establishes a hydrostatic pressure that pushes the liquid medium from the liquid medium reservoir, vertically through the filter membrane, and into the sample well where it permeates the 3D cell growth medium, and
wherein when the liquid medium reservoir is filled to the first volume, the filter membrane has a pore size configured to produce a perfusion velocity of the liquid medium through the 3D cell growth medium at from 250 nm/s to 5 μm/s.

2. The system of claim 1, wherein the filter membrane has a pore size from 100 nm to 10 μm.

3. The system of claim 1, further comprising a means for transferring the liquid medium from the sample well to the liquid medium reservoir.

4. The system of claim 1, further comprising an overflow lip or drain in the sample well configured to empty the liquid medium into a collection container, thereby maintaining a constant sample well volume.

5. The system of claim 4, further comprising a means for transferring the liquid medium from the collection container to the liquid medium reservoir.

6. The system of claim 5, wherein the means for transferring the liquid medium from the collection container to the liquid medium reservoir is a peristaltic pump.

7. The system of claim 3, further comprising a means for monitoring the volume of liquid medium in the liquid medium reservoir configured to trigger the means for transferring the liquid medium from the collection container to the liquid medium reservoir when the volume of liquid medium in the liquid medium reservoir drops below the first volume.

8. The system of claim 7, wherein the means for monitoring the volume of liquid medium in the liquid medium reservoir is an optical sensor or float switch.

9. The system of claim 1, comprising an array of isolated sample wells that are all fluidly connected to the same liquid medium reservoir.

10. The system of claim 1, comprising an array of isolated sample wells that are each fluidly connected to a separate liquid medium reservoir.

11. The system of claim 1, wherein the bottom of the sample well is optically transparent.

12. The system of claim 1, wherein the 3D cell culture medium is a hydrogel.

13. The system of claim 12, wherein the 3D cell culture medium comprises a plurality of hydrogel particles and a liquid cell culture medium, wherein the hydrogel particles are swelled with the liquid cell culture medium to form a granular gel.

14. The system of claim 13, wherein the 3D cell culture medium has a yield stress such that the cell growth medium undergoes a phase change from a first solid phase to a second liquid phase upon application of a shear stress greater than the yield stress.

15. The system of claim 14, wherein the yield stress is on the order of 10 Pa.

16. The system of claim 13, wherein the concentration of hydrogel particles is between 0.05% to about 1.0% by weight.

17. The system of claim 13, wherein the hydrogel particles have a size between about 0.1 μm to about 100 μm when swollen with the liquid cell culture medium.

18. The system of claim 1, wherein the 3D cell culture medium is a liquid medium.

19. The system of claim 1, wherein a plurality of cells are disposed in the 3D cell culture medium.
